(19) United States
(12) Patent Application Publication
(10) Pub. No.: US 2026/0279597 A1
(43) Pub. Date: Sep. 17, 2026

(54) Internet-Based Infectious Disease Prevention and Control Training System and Method

(71) Applicant: The Hong Kong Polytechnic University, Hong Kong (CN)

(72) Inventors: Yee Man Angela LEUNG, Hong Kong (CN); Terence Kenneth LAU, Hong Kong (CN); Alexandros MOLASIOTIS, Hong Kong (CN); Yat Pang CHEUNG, Hong Kong (CN)

(21) Appl. No.: 19/162,534

(22) PCT Filed: Feb. 6, 2024
(86) PCT No.: PCT/CN2024/076227

§ 371 Date: Sep. 5, 2025

(30) Foreign Application Priority Data
Mar. 8, 2023 (CN) 202310214233.0

(51) Int. Cl. G16H 50/80; A63F 13/80; G06F 21/31

(52) U.S. Cl. CPC G16H 50/80; A63F 13/80; G06F 21/31

(57) ABSTRACT

An Internet-based infectious disease prevention and control training system and an Internet-based infectious disease prevention and control training method. The system comprises a resource database, an application server, a network medium, and an application program on an intelligent terminal; the resource database comprises a service database and a user information database; the service database comprises texts, audios, images, short videos, and game controls; the application server comprises an access management module, a resource scheduling module, a report generation module, and a network optimization module; the access management module authenticates, on the basis of the user information database, login request information sent by a user; the resource scheduling module schedules the texts, the audios, the images, the short videos, or the game controls in the service database according to an instruction sent by the application program, and presents the texts, the audios, the images, the short videos, or the game controls to the intelligent terminal by means of the network medium; the application program comprises a plurality of topics associated with an infectious disease prevention and control normative document; and each topic is presented in the form of one or more games.

### TECHNICAL FIELD

The present invention relates to Internet-based infectious disease prevention and control training system and method, which are presented in the form of games.

### BACKGROUND

Infectious disease prevention and control is a core factor for curb the spreading of pathogen and virus infection. In Hong Kong, there are more than 400 nursing homes. During the COVID-19 epidemic, the elderly in the nursing homes became COVID-19 susceptible population and infected high-risk population. According to government data, in the fifth wave of the COVID-19 epidemic, about 55% of deaths occurred in nursing homes and homes for the disabled, and more than 95% of the deaths were elderly people aged 60 or above. As a result, the public pay more and more attention to how the elderly were infected in the nursing homes, whether there are enough measures to prevent and control infectious diseases in the nursing homes, and whether the nursing staff in the nursing homes have enough experience and are well-trained to make preparation for the prevention and control of infectious diseases.

During the epidemic period, which was different from usual, higher requirements were put forward for the nursing staff in the nursing homes: the nursing staff should prevent the elderly and the nursing staff themselves from being infected by viruses in addition to providing daily care for the elderly. The nursing staff faced a higher risk of potential virus infection in their efforts to protect the community. They might be confronted with psychological distress, fatigue, job burnout or humiliation, etc. Effective training for infectious disease prevention and control can reduce the risk of infection, effectively protect the nursing staff, and prevent the elderly from being infected by viruses.

At present, there is no effective training system for the nursing staff in nursing homes in the market. Usually, the recommended infectious disease prevention and control manual has ten to dozens of pages, and the content of the manual is boring. Consequently, it is difficult for the nursing staff in nursing homes to achieve an effective training effect by reading the infectious disease prevention and control manual directly. Even if the prevention and control manual is reduced to leaflets, the amount of information is large and the content is loose. Therefore, it is difficult for the nursing staff to keep an effective memory after reading the leaflets, especially during an epidemic time, in which the nursing staff in nursing homes often bear great psychological pressure. Studies have shown that the learning ability of people who work under great pressure will be significantly compromised.

The inventors of the present invention have developed Internet-based infectious disease prevention and control training system and method, which can be used to train the nursing staff in nursing homes systematically and pertinently, such that they can learn and understand various steps and measures to be taken in different infectious disease prevention and control scenarios in a manner like playing games. The Internet-based infectious disease prevention and control training system in the present invention is presented to the nursing staff in nursing homes in a multimedia and game interaction way. It improves the participation of the nursing staff in the training, and has won unanimous praise from the nursing staff who participated in evaluations. The present invention provides a safe and flexible learning environment for the nursing staff in nursing homes to learn and practice decision-making.

### SUMMARY OF THE INVENTION

The object of the present invention is to provide an Internet-based infectious disease prevention and control training system, which comprises a resource database, an application server, a network medium, and an application program on an intelligent terminal, wherein: the resource database comprises a service database and a user information database, and the service database includes texts, audios, images, short videos and game controls; the application server comprises an access management module, a resource scheduling module, a report generation module, and a network optimization module, wherein the access management module authenticates login request information sent by a user via the application program based on the user information database, the resource scheduling module schedules the texts, audios, images, short videos, or game controls in the service database according to instructions sent via the application program, and presents the texts, audios, images, short videos, or game controls to the intelligent terminal through the network medium; and the application program includes a plurality of topics related to an infectious disease prevention and control normative document, and each of the topics is presented in the form of one or more games.

In some embodiments, the one or more games include texts, images, audios, short videos, and simulations of different roles and application scenarios.

In some embodiments, the one or more games simulate infectious disease prevention and control scenarios under different topics, such that the user can practice repeatedly in simulated infectious disease prevention and control scenarios.

In some embodiments, the plurality of topics includes Preparation, Emergency Response, Self-Protection, Application of Protective Equipment, Communication, Isolating Viruses Outdoor, Stress Management, and Five Moments of Hand Washing.

In some embodiments, the infectious disease prevention and control scenarios include one or more of the following items: visitor consultation and information logging; different treatment measures for suspected cases and confirmed cases; guidelines and methods for environmental disinfection; arrangement of social distance; disinfection methods for various articles; placement of hand rubbing liquid; screening of personal protective equipment in different scenarios; selection of personal protective equipment; timing, frequency and time required for cleaning hands; selection of cleaning liquid; hand washing steps; nursing steps; methods for communication with confirmed patients; and ways for the nursing staff to relieve stress.

In some embodiments, the infectious disease prevention and control normative document is issued by the World Health Organization (WHO) or the Department of Health of Hong Kong.

In some embodiments, the one or more games include voice prompts for guiding the operation of the user.

In some embodiments, the user comprises nursing staff, administrative staff, elderly people living in nursing homes and visiting family members.

In some embodiments, the user information database records login time of the user, time spent for completing the training under each topic, whether the training under each topic is completed, number of times of participation, and scores, wherein account name and password of the user are uniquely defined.

In some embodiments, in each game scenario, a progress of the game under the topic is prompted to the user.

In some embodiments, the user can continue the game at the progress at the next time of login.

In some embodiments, the report generation module generates statistical information of the user based on the login time of the user, the time spent for completing the training under each topic, whether the training under each topic is completed, the number of times of participation and the scores recorded in the user information database, and the statistical information of the user includes online training time, training progress, overall rating, and feedback and suggestions.

In some embodiments, an administrator evaluates a training status of the user based on the statistical information of the user.

In some embodiments, the user is permitted to see the evaluation and rating.

The present invention further provides an Internet-based infectious disease prevention and control training method, which comprises the following steps: sending login request information, by a user, to an application server via an application program on an intelligent terminal through a network medium, authenticating the login request information, by an access management module of the application server, based on a user information database of a resource database, and scheduling texts, audios, images, short videos or game controls in a service database of the resource database according to instructions sent via the application program, and presenting the texts, audios, images, short videos or game controls to the intelligent terminal through the network medium, by a resource scheduling module of the application server, wherein the application program includes a plurality of topics related to an infectious disease prevention and control normative document, and each of the topics is presented in the form of one or more games.

Other features and advantages of the present invention will become apparent from the following detailed description, accompanying drawings and claims.

### BRIEF DESCRIPTION OF THE DRAWINGS

The forementioned and additional features of the present invention will become apparent from the following description of exemplary preferred embodiments provided in conjunction with the accompanying drawings, in which:

FIG. 1 shows a schematic block diagram of an Internet-based infectious disease prevention and control training system according to the embodiments of the present invention;

FIG. 2 shows a main interface presented by an intelligent terminal according to the embodiments of the present invention;

FIG. 3 shows a visitor registration scenario under a topic of “Preparation” according to the embodiments of the present invention;

FIG. 4 shows a scenario of explanation made by a head nurse under a topic of “Self-Protection” according to the embodiments of the present invention;

FIG. 5 shows a scenario of screening protective equipment for suspected or confirmed cases under a topic of “Application of Protective Equipment” according to the embodiments of the present invention;

FIG. 6 shows a scenario of comforting a confirmed patient by a nurse under a topic of “Communication” according to the embodiments of the present invention;

FIG. 7 shows a scenario of environment disinfection under a topic of “Isolating Viruses Outdoor” according to the embodiments of the present invention;

FIG. 8 shows a scenario of stress relief for the nursing staff under a topic of “Stress Management” according to the embodiments of the present invention;

FIG. 9 shows a scenario of interactive game for specific nursing steps under a topic of “Five Moments of Hand Washing” according to the embodiments of the present invention;

FIG. 10 shows a schematic composition diagram of the user information database according to the embodiments of the present invention;

FIG. 11 shows a schematic composition diagram of the service database according to the embodiments of the present invention; and

FIGS. 12 and 13 show test results of the effectiveness of the infectious disease prevention and control training system according to the embodiments of the present invention.

### DETAILED DESCRIPTION OF THE INVENTION

In the following claims and the above description of the present invention, the term “comprise” or its variants (e.g., “include” or “contain”) are used in an inclusive manner, i.e. it indicates the presence of the stated features, but does not exclude the presence or addition of additional features in various embodiments of the present invention, unless the context specifies otherwise owing to the expressive language or necessary implication.

It should be understood that the reference to any prior art publication herein does not constitute an admission that the publication is part of the common knowledge in any country and in the art.

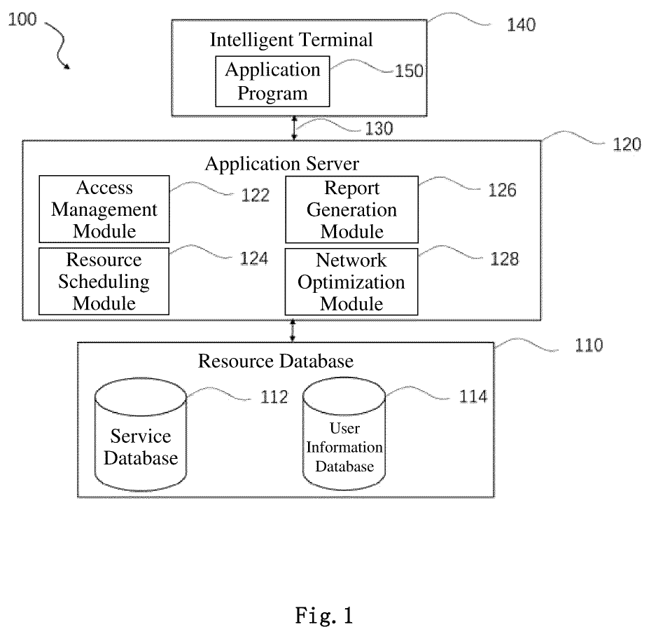

FIG. 1 shows a schematic block diagram of an Internet-based infectious disease prevention and control training system 100 according to the embodiments of the present invention. The infectious disease prevention and control training system 100 comprises a resource database 110, an application server 120, a network medium 130, and an application program 150 on an intelligent terminal 140. The resource database 110 comprises a service database 112 and a user information database 114; and the application server 120 comprises an access management module 122, a resource scheduling module 124, a report generation module 126 and a network optimization module 128.

The application server 120 and the intelligent terminal 140 may be in a typical client-server architecture. The intelligent terminal 140 includes, but is not limited to, a personal computer or mobile communication device (e.g., a mobile phone, a tablet device, etc.) based on an iOS operating system or Android operating system. The network medium 130 may be any wired or wireless communication medium, including, but not limited to a telephone line, an optical fiber, GPRS, CDMA, WiFi, and Bluetooth, etc. Next, a mobile phone is used as an example of the intelligent terminal 140.

First, a user opens a main interface of the infectious disease prevention and control training system 100 via an application program 150 on the mobile phone, and inputs a user name and a password to request for login. For different users, the user names and passwords are uniquely defined. The user names and passwords may be user names and passwords pre-assigned to the nursing staff in each nursing home. The access management module 122 located at the application server 120 searches in the user information database 114. If the user name and password inputted by the user match the user information stored in the user information database 114, a login success message will be sent to the user.

In view that the infectious disease prevention and control training system 100 is mainly oriented to the nursing staff in nursing homes, preferably user names and passwords pre-assigned to the nursing staff are employed. Alternatively, in other embodiments, a user registration approach may be used, the identities of the nursing staff may be verified during the user registration, and the users may set their user names and passwords, which will be stored in the user information database 114.

If the user name and password inputted by the user do not match the user information stored in the user information database 114, a message containing login failure and help information will be sent to the user. Alternatively, in some embodiments, a mobile phone number in the user information stored in the user information database 114 may be used to log in by sending a one-time password to the mobile phone number. In some other embodiments, the user is permitted to change his/her login password after the one-time password is verified via the mobile phone number.

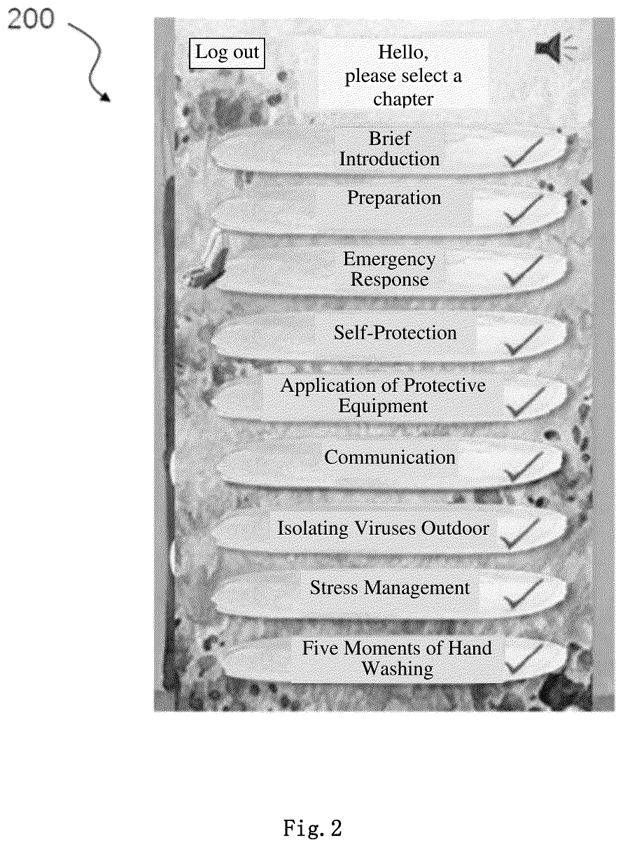

After successful login, the intelligent terminal 140 presents a main interface 200, for example, as shown in FIG. 2. According to the normative document for infectious disease prevention and control of COVID-19 provided by the World Health Organization (WHO) and the Department of Health of Hong Kong, the infectious disease prevention and control training system 100 mainly provides eight topics: Preparation, Emergency Response, Self-Protection, Application of Protective Equipment, Communication, Isolating Viruses Outdoor, Stress Management, and Five Moments of Hand Washing. In order to mobilize the nursing staff to participate in the training and improve the training effect, each topic is presented in the form of one or more mini-games. Specifically, the resource scheduling module 124 schedules texts, audios, images, short videos or game controls in the service database 112 according to the instructions sent via the application program 150, and presents corresponding texts, audios, images, short videos or game controls to the intelligent terminal 140 through the network medium 130. The mini-games under each topic may include texts, images, audios, short videos, and simulations of different roles and application scenarios. The mini-games include voice prompts for guiding the operation of the user. Different games can simulate infectious disease prevention and control scenarios under different topics, so that the user can practice repeatedly in simulated infectious disease prevention and control scenarios. The infectious disease prevention and control scenarios at least include one or more of the following items: visitor consultation and information logging; different treatment measures for suspected cases and confirmed cases; guidelines and methods for environment disinfection; arrangement of social distance; disinfection methods for various articles; placement of hand rubbing liquid; screening of personal protective equipment in different scenarios; selection of personal protective equipment; timing, frequency and time required for cleaning hands; selection of cleaning liquid; hand washing steps; nursing steps; methods for communication with confirmed patients; and ways for the nursing staff to relieve stress.

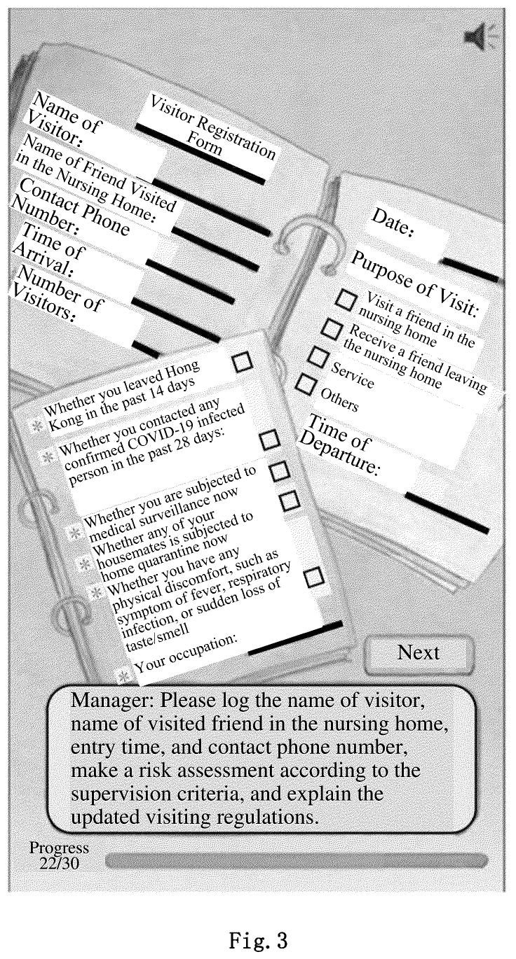

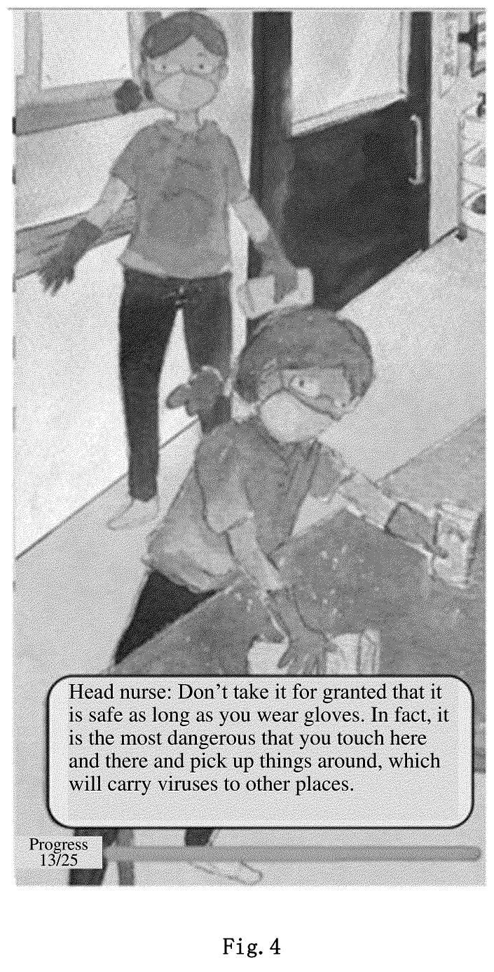

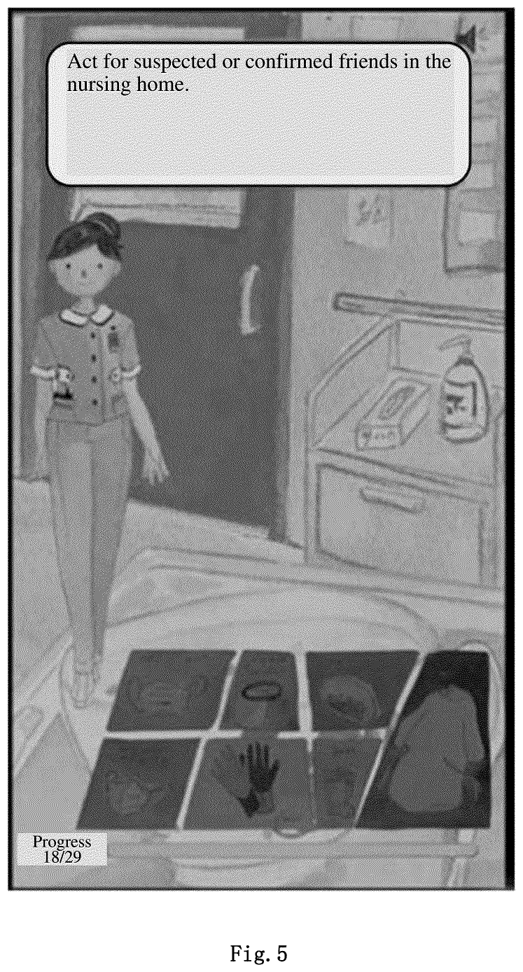

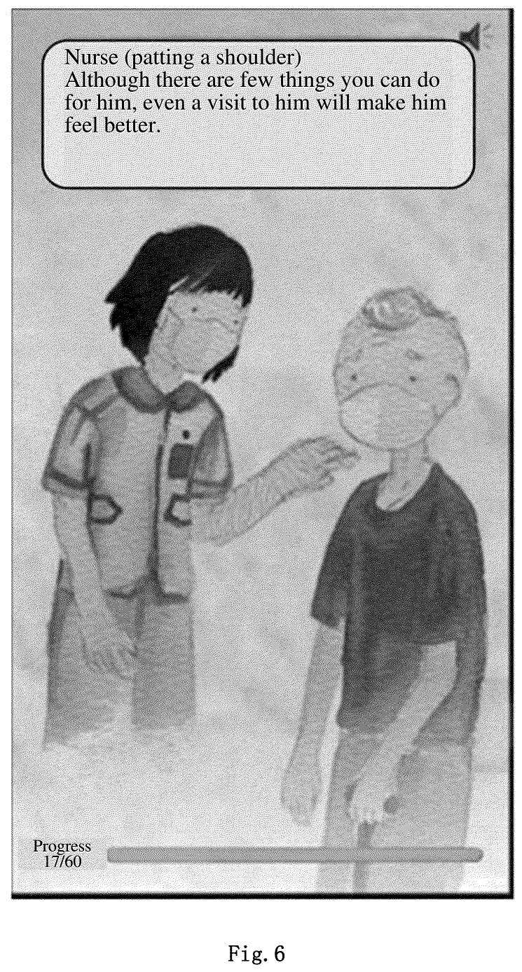

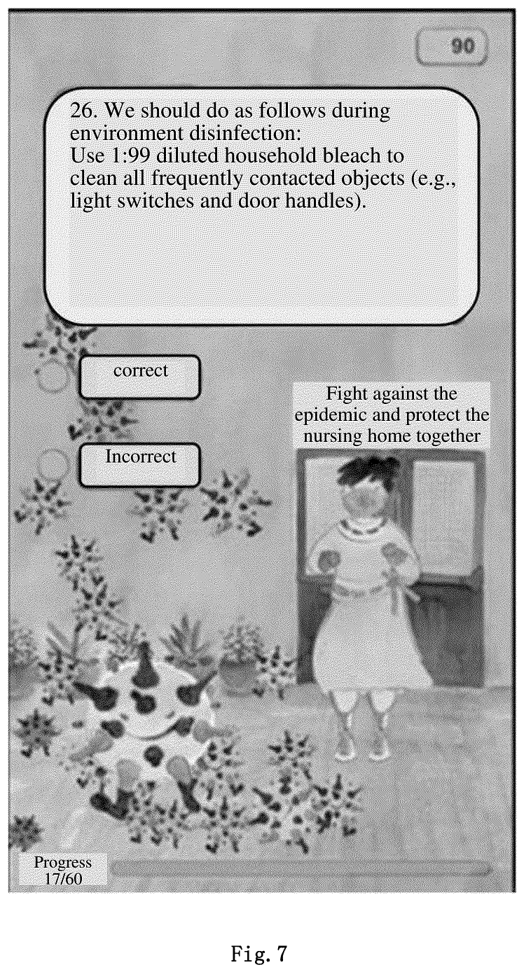

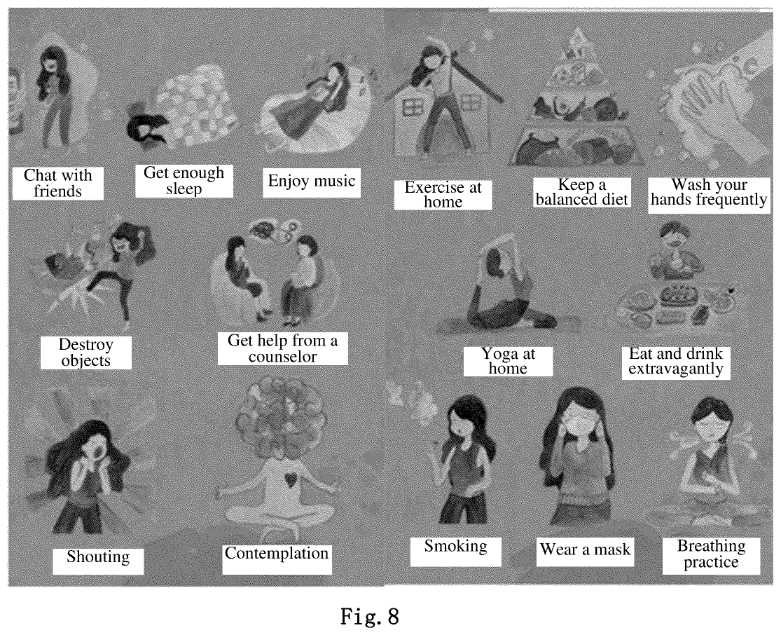

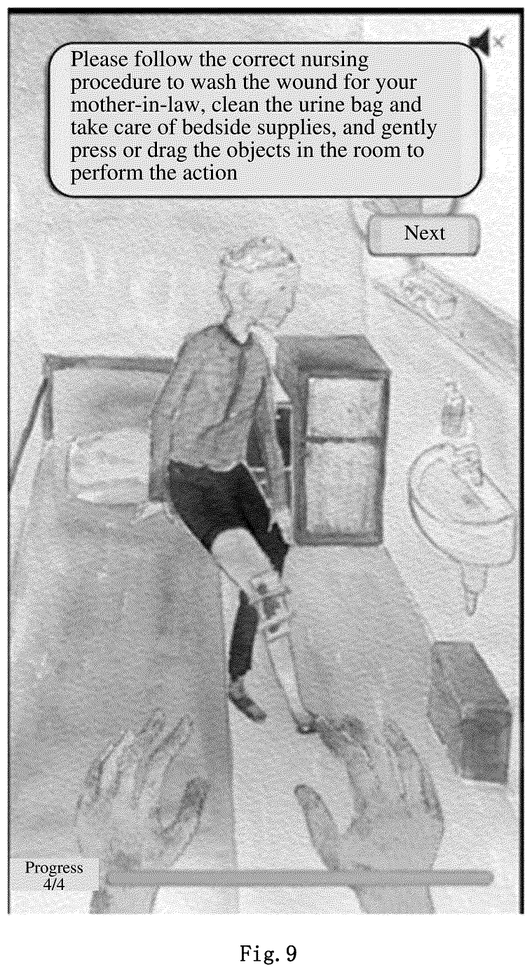

FIGS. 3 to 9 show exemplary infectious disease prevention and control scenarios according to the embodiments of the present invention. FIG. 3 shows a scenario of visitor consultation and information logging under a topic of “Preparation”. FIG. 4 shows a scenario of explanation made by a head nurse under a topic of “Self-Protection”. FIG. 5 shows a scenario of screening protective equipment for suspected or confirmed cases under a topic of “Application of Protective Equipment”. FIG. 6 shows a scenario of comforting a confirmed patient by a nurse under a topic of “Communication”. FIG. 7 shows a scenario of environment disinfection under a topic of “Isolating Viruses Outdoor”. The topic “Isolating Viruses Outdoor” includes 40 multiple-choice questions, which are provided with pictures and scenarios in combination to consolidate memory. FIG. 8 shows a scenario of stress relief for the nursing staff under a topic of “Stress Management”. FIG. 9 shows an interactive game scenario of specific nursing steps under a topic of “Five Moments of Hand Washing”. The interactive game shown in FIG. 9 simulates a real scenario, so that the user can practice repeatedly by means of gestures in the simulated infectious disease prevention and control scenario. The gestures may include clicking, dragging and other operations. In some embodiments, the operations may be practiced with the help of a gravity sensor, an acceleration sensor, a gyroscope, and other sensors of the intelligent terminal. The inventors set up different game forms based on the knowledge on prevention and control of different infectious diseases and present them to the nursing staff in the most appropriate way.

FIG. 10 shows a schematic composition diagram of the user information database 114 according to the embodiments of the present invention. In addition to user name, password, nursing home, and contact information, the user information of each user further includes the user's login time, time required to complete the training under each topic, whether the training under each topic is completed, number of times of participation, and scores. Thus, in each game scenario, the user can be prompted about the progress of the game under the topic. For example, as shown in FIG. 7, a progress bar is presented at the bottom of the game page to effectively prompt the user about the progress under the topic. FIG. 7 shows that the user is answering the 26^{th} question among 40 multiple-choice questions. Furthermore, the user can continue the game at the progress when he/she logs in at the next time, so as to save the time for the user. The nursing staff participating in the training is encouraged to complete the training under each topic independently within 15 minutes, and complete the training under 8 topics within 2 weeks, which takes about 120 minutes in total.

The users of the infectious disease prevention and control training system 100 may further include administrators of nursing homes, elderly people living in nursing homes and visiting family members, in addition to the nursing staff. The administrators may view and evaluate the training status of the employees at any time. Specifically, the report generation module 126 generates statistical information of the user based on the login time of the user, the time spent for completing the training under each topic, whether the training under each topic is completed, the number of times of participation and the scores recorded in the user information database 114, and the statistical information of the user may include online training time, training progress, overall rating, and feedback and suggestions. Each user participating in the training is permitted to see the evaluation and rating under each topic.

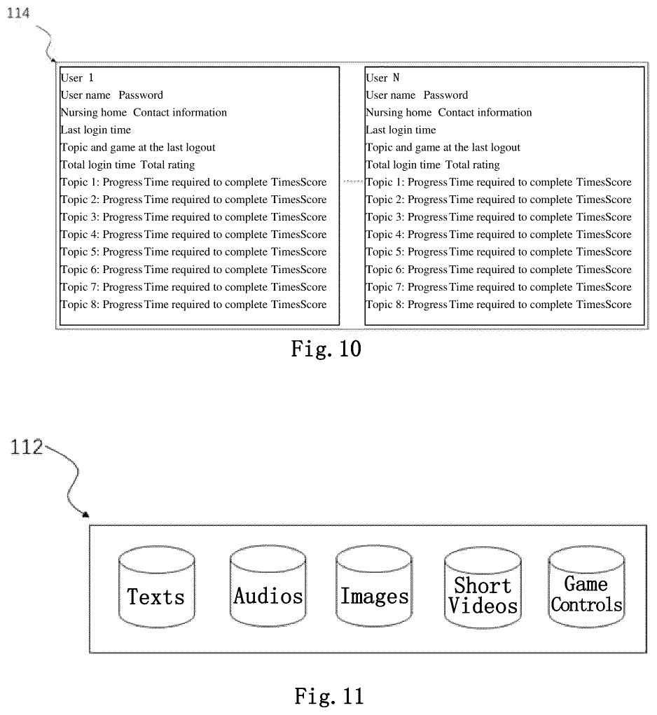

FIG. 11 shows a schematic composition diagram of the service database 112 according to the embodiments of the present invention. In the development of the present invention, the service database 112 has accumulated rich resources of texts, audios, images, short videos and game controls. In order to facilitate system migration or secondary development and simplify the work of developers, the resource data may be indexed by tags, and natural language semantic analysis may be carried out on new rules in current normative documents or new normative documents, and text descriptions may be quickly matched with the tags to automatically locate corresponding resources, so as to invoke, replace or update the resources and improve the screening efficiency. In addition, in some embodiments, the scenarios or roles may be replaced with scenarios or roles in specific nursing homes to improve friendliness or interest. Furthermore, in some embodiments, different game types may be defined to facilitate reuse, so that the developers do not have to rewrite the codes for duplicated game types (e.g., multiple-choice questions, true-false questions, etc.), whereby the labor of the developers is reduced.

The inventors conducted extensive tests on the effectiveness of the infectious disease prevention and control training system 100, and selected 114 persons in an intervention group (IG) and 98 persons in a control group (CG), which were distributed in 13 nursing homes. The IG was trained with the infectious disease prevention and control training system 100, while the CG was trained in the traditional form, and comparative statistics was performed on the performance of specific practical items before and after the training.

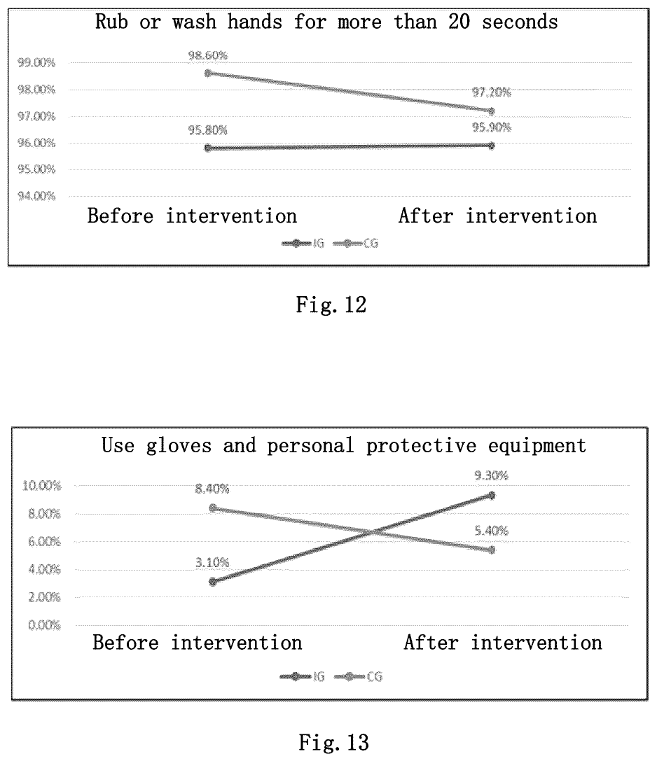

FIG. 12 shows the comparison between the IG and the CG in hand rubbing and hand washing. It can be seen that the time of rubbing hands or washing hands in the IG was improved after using the infectious disease prevention and control training system 100, while the performance of the CG exhibited a slight compromised trend. The test was started when the epidemic situation was serious. At that time, the awareness of hand rubbing and hand washing of the nursing staff was significantly improved. The epidemic situation became alleviated slightly after two weeks of training in the IG, so the performance of the CG exhibited a compromised trend. In contrast, the performance of the IG still maintained an improving trend after the training. FIG. 13 shows the comparison between the IG and the CG in the use of gloves and personal protective equipment. It can be seen that the performance of the IG was significantly improved after the training, while the performance of the CG was lagged behind.

Through the test, it was proved that the infectious disease prevention and control training system 100 was effective. After the test, the inventors got feedback from the users who participated in the training., The users talked about the experience in using the infectious disease prevention and control training system 100 for training: 1) the training duration is short, and the automation of the training is the most favored; 2) the training is provided in an online mode, and the user can operate the system personally; 3) the system is simple and easy to use, and members at all levels can use the system easily, regardless of their education levels; 4) a literal document is transformed into interactive games and short videos, which are more interesting, easy to remember and improve participation.

The gamified training has many advantages. Conventional boring experience is reorganized into interesting, interactive and competitive scenario simulation, so that users can keep an active learning status and participate in their training actively. In the training system developed in the present invention, the users can gain experience on decision-making, multitasking and cooperation with others. For example, the infectious disease prevention and control training system 100 may include an intelligent terminal collaboration management module to support collaborative operation of a plurality of intelligent terminals 140. The network optimization module 128 can ensure the best user experience with the intelligent terminals 140 under different network conditions.

The Internet-based infectious disease prevention and control training system 100 in the present invention allows users to learn at their own pace at any convenient time and place. They can learn according to their own schedules. The infectious disease prevention and control training system 100 reduces the number of trainers required to provide necessary training, and makes the training courses highly consistent with each other.

While the present invention is illustrated and described above in detail in some embodiments with reference to the accompanying drawings, the above embodiments are only illustrative rather than limitative. The present invention further encompasses various combinations, modifications and variations of the exemplary embodiments made without departing from the spirit and scope of the present invention.

What is claimed is:

1. An Internet-based infectious disease prevention and control training system, comprising a resource database, an application server, a network medium, and an application program on an intelligent terminal, wherein:
the resource database comprises a service database and a user information database, wherein the service database includes texts, audios, images, short videos, and game controls;
the application server comprises an access management module, a resource scheduling module, a report generation module, and a network optimization module,
wherein the access management module authenticates login request information sent by a user via the application program based on the user information database, the resource scheduling module schedules the texts, audios, images, short videos, or game controls in the service database according to instructions sent via the application program, and presents the texts, audios, images, short videos, or game controls to the intelligent terminal through the network medium; and
wherein the application program includes a plurality of topics related to an infectious disease prevention and control normative document, and each of the topics is presented in the form of one or more games.

2. The infectious disease prevention and control training system according to claim 1, wherein the one or more games include texts, images, audios, short videos, and simulations of different roles and application scenarios.

3. The infectious disease prevention and control training system according to claim 1, wherein the one or more games simulate infectious disease prevention and control scenarios under different topics, so that the user is able to practice repeatedly in simulated infectious disease prevention and control scenarios.

4. The infectious disease prevention and control training system according to claim 1, wherein the plurality of topics includes Preparation, Emergency Response, Self-Protection, Application of Protective Equipment, Communication, Isolating Viruses Outdoor, Stress Management, and Five Moments of Hand Washing.

5. The infectious disease prevention and control training system according to claim 3, wherein the infectious disease prevention and control scenarios include one or more of the following items: visitor consultation and information logging; different treatment measures for suspected cases and confirmed cases; guidelines and methods for environmental disinfection; arrangement of social distance; disinfection methods for various articles; placement of hand rubbing liquid; screening of personal protective equipment in different scenarios; selection of personal protective equipment; timing, frequency and time required for cleaning hands; selection of cleaning liquid; hand washing steps; nursing steps; methods for communication with confirmed patients; and ways for the nursing staff to relieve stress.

6. The infectious disease prevention and control training system according to claim 1, wherein the infectious disease prevention and control normative document is issued by the World Health Organization (WHO) or the Department of Health of Hong Kong.

7. The infectious disease prevention and control training system according to claim 1, wherein the one or more games include voice prompts for guiding the operation of the user.

8. The infectious disease prevention and control training system according to claim 1, wherein the user comprises nursing staff, administrative staff, elderly people living in nursing homes and visiting family members.

9. The infectious disease prevention and control training system according to claim 1, wherein the user information database records login time of the user, time spent for completing the training under each topic, whether the training under each topic is completed, number of times of participation, and scores, wherein account name and password of the user are uniquely defined.

10. The infectious disease prevention and control training system according to claim 1, wherein in each game scenario, a progress of the game under the topic is prompted to the user.

11. The infectious disease prevention and control training system according to claim 10, wherein the user can continue the game at the progress at the next time of login.

12. The infectious disease prevention and control training system according to claim 8, wherein the report generation module generates statistical information of the user based on the login time of the user, the time spent for completing the training under each topic, whether the training under each topic is completed, the number of times of participation and the scores recorded in the user information database, and the statistical information of the user includes online training time, training progress, overall rating, and feedback and suggestions.

13. The infectious disease prevention and control training system according to claim 12, wherein an administrator evaluates a training status of the user based on the statistical information of the user.

14. The infectious disease prevention and control training system according to claim 1, wherein the user is permitted to see the evaluation and rating.

15. An Internet-based infectious disease prevention and control training method, comprising the following steps:
sending login request information, by a user, to an application server via an application program on an intelligent terminal through a network medium,
authenticating the login request information, by an access management module of the application server, based on a user information database of a resource database, and
scheduling texts, audios, images, short videos or game controls in a service database of the resource database according to instructions sent via the application program, and presenting the texts, audios, images, short videos or game controls to the intelligent terminal through the network medium, by a resource scheduling module of the application server,
wherein the application program includes a plurality of topics related to an infectious disease prevention and control normative document, and each of the topics is presented in the form of one or more games.

16. The infectious disease prevention and control training method according to claim 15, wherein the one or more games include texts, images, audios, short videos, and simulations of different roles and application scenarios.

17. The infectious disease prevention and control training method according to claim 15, wherein the one or more games simulate infectious disease prevention and control scenarios under different topics, so that the user can practice repeatedly in simulated infectious disease prevention and control scenarios.

18. The infectious disease prevention and control training method according to claim 15, wherein the plurality of topics includes Preparation, Emergency Response, Self-Protection, Application of Protective Equipment, Communication, Isolating Viruses Outdoor, Stress Management, and Five Moments of Hand Washing.

19. The infectious disease prevention and control training method according to claim 17, wherein the infectious disease prevention and control scenarios include one or more of the following items: visitor consultation and information logging; different treatment measures for suspected cases and confirmed cases; guidelines and methods for environmental disinfection; arrangement of social distance; disinfection methods for various articles; placement of hand rubbing liquid; screening of personal protective equipment in different scenarios; selection of personal protective equipment; timing, frequency and time required for cleaning hands; selection of cleaning liquid; hand washing steps; nursing steps; methods for communication with confirmed patients; and ways for the nursing staff to relieve stress.

20. The infectious disease prevention and control training method according to claim 15, wherein the infectious disease prevention and control normative document is issued by the World Health Organization (WHO) or the Department of Health of Hong Kong.

21. The infectious disease prevention and control training method according to claim 15, wherein the one or more games include voice prompts for guiding the operation of the user.

22. The infectious disease prevention and control training method according to claim 15, wherein the user comprises nursing staff, administrative staff, elderly people living in nursing homes and visiting family members.

23. The infectious disease prevention and control training method according to claim 15, wherein the user information database records login time of the user, time spent for completing the training under each topic, whether the training under each topic is completed, number of times of participation, and scores, wherein account name and password of the user are uniquely defined.

24. The infectious disease prevention and control training method according to claim 15, wherein in each game scenario, a progress of the game under the topic is prompted to the user.

25. The infectious disease prevention and control training method according to claim 24, wherein the user can continue the game at the progress at the next time of login.

26. The infectious disease prevention and control training method according to claim 23, wherein the report generation module of the application server generates statistical information of the user based on the login time of the user, the time spent for completing the training under each topic, whether the training under each topic is completed, the number of times of participation and the scores recorded in the user information database, and the statistical information of the user includes online training time, training progress, overall rating, and feedback and suggestions.

27. The infectious disease prevention and control training method according to claim 26, wherein an administrator evaluates a training status of the user based on the statistical information of the user.

28. The infectious disease prevention and control training method according to claim 15, wherein the user is permitted to see the evaluation and rating.
